(19) United States
(12) Patent Application Publication
(10) Pub. No.: US 2011/0082197 A1
(43) Pub. Date: Apr. 7, 2011

(54) Method for inhibiting formation and/or activation of osteoclasts using flemingia macrophylla extract or lespedeza flavanone A

(75) Inventors: Jin-Bin Wu, Taichung (TW); Wen-Chuan Lin, Taichung (TW); Hui-Ya Ho, Taichung (TW)

(73) Assignee: CHINA MEDICAL UNIVERSITY, Taichung (TW)

(21) Appl. No.: 12/661,656

(22) Filed: Mar. 22, 2010

(30) Foreign Application Priority Data
Oct. 7, 2009 (TW) 098133938

(51) Int. Cl. A61K 31/352; A61P 19/00; A61P 19/10

(52) U.S. Cl. 514/456

(57) ABSTRACT

A method for inhibiting the formation and/or activation of osteoclasts in a mammal comprising administrating an effective amount of a compound of formula (I) or a pharmaceutically acceptable salt or ester thereof to the mammal is provided:

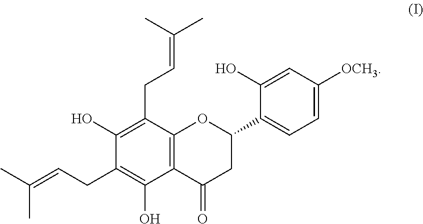

Also provided is a method for the preparation of a Flemingia macrophylla extract.

### CROSS-REFERENCES TO RELATED APPLICATIONS

This application claims the benefit of Taiwan Patent Application No. 098133938, filed on Oct. 7, 2009, in the Taiwan Intellectual Property Office, the disclosure of which is incorporated herein in its entirety by reference.

### BACKGROUND OF THE INVENTION

1. Field of the Invention

The present invention relates to the uses of Lespedeza flavanone A (LDF-A) in the inhibition of the formation and/or activation of osteoclasts.

2. Descriptions of the Related Art

Bones mainly consist of organic components (e.g., collagenic fibers and mucopolysaccharides), inorganic components (e.g., calcium salts and phosphor salts) and water. Bones are tissues that exist in a dynamic equilibrium state with a “bone remodeling” process, in which bone formation and bone resorption occur constantly. This process is not only responsible for recovering from slight trauma, but also enhances the pressure resistance of bones. The bone formation includes the formation of new bone matrices and the mineralization of bones.

The bone remodeling process relies on the cooperation between two different kinds of cells, osteoblasts and osteoclasts, responsible for bone formation and bone resorption, respectively. If any mistake occurs in the coordination between the two cells, an imbalance of the bone remodeling process may arise. For example, osteoporosis, commonly seen in clinical medicine (especially in postmenopausal women with insufficient secretion of estrogen), occurs if the level of bone resorption is higher than that of bone formation. On the other hand, if the level of bone resorption is lower than that of the bone formation, which is rare, an abnormal increase of bone tissues may occur.

Currently, there are about two hundred million females suffering from osteoporosis in the world. In 2003, the global market for osteoporosis and hormone supplement therapy for osteoporosis was worth about 8.3 billion USD, and is predicted to reach 17.9 billion USD in 2014. Pharmaceuticals for osteoporosis can be generally classified into four groups based on the mechanisms. The first group inhibits bone resorption, an example of which is diphosphates; the second group stimulates bone formation, an example of which is parathyroid hormone; the third group inhibits the release of calcium from bones, an example of which is estrogen; and the fourth group stimulates the small intestine to absorb calcium, an example of which is Vitamin D. However, diphosphates may lead to strong side effects, such as headaches, nausea, vomiting, diarrhea, fever, renal failure, oesophagitis, mandible necrosis, etc. Parathyroid hormone may cause discomfort, such as headaches and nausea. Estrogen has a risk of causing cancer. In addition, the effect of using Vitamin D to enhance the absorption of calcium to improve osteoporosis is quite limited. Therefore, approaches efficiently treating osteoporosis with minimal side effects are still desired.

It is known that Flemingia macrophylla belonging to Leguminosae plants has effects of anti-osteoporosis, reducing blood sugar, anti-rheumatoid arthritis, etc. (see Syiem et al, 2007, Evaluation of Flemingia macrophylla L., a traditionally used plant of the north eastern region of India for hypoglycemic and anti-hyperglycemic effect on mice. Pharmacology online, 2: 355-66, which is entirely incorporated hereinto by reference). Nevertheless, in terms of the anti-osteoporosis effect, active components in Flemingia macrophylla remain unclear at present, and related research may only focus on the crude extracts of Flemingia macrophylla, and thus, the optimization of drug efficiency and the pharmacological study are limited. Accordingly, if active components of Flemingia macrophylla can be obtained, further pharmacological trials can be carried out, and approaches efficiently treating osteoporosis with minimal side effects can be provided.

The present invention is the investigation for the above requirements. The inventors of the present invention discovered a main active compound for anti-osteoporosis in Flemingia macrophylla through related in vivo and in vitro experiments, and found that a Flemingia macrophylla extract has a function of inhibiting bone resorption.

### SUMMARY OF THE INVENTION

One objective of this invention is to provide a method for inhibiting the formation and/or activation of osteoclasts in a mammal comprising administrating an effective amount of a compound of formula (I) or a pharmaceutically acceptable salt or ester thereof to the mammal:

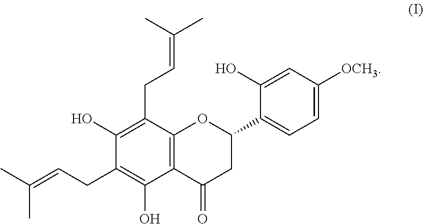

Another objective of this invention is to provide a method for the preparation of a Flemingia macrophylla extract comprising the compound of formula (I).

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 illustrates microscopic graphs of the trabecular bone and the dermal bone of the metaphysis of the tibia of a Wistar rat;

FIG. 2 illustrates microscopic graphs of osteoclasts around the trabecular bone of the tibia of a Wistar rat;

FIG. 3 illustrates a flow chart for extracting and isolating Lespedeza flavanone A; and

FIG. 4 is a diagram illustrating the mechanism of Lespedeza flavanone A.

### DESCRIPTION OF THE PREFERRED EMBODIMENT

Osteoporosis is caused by the imbalance between bone formation (from osteoblasts) and bone resorption (from osteoclasts). Specifically, one of causes for osteoporosis is that the number of osteoclasts in bones is much greater than normal, or the rate of the bone resorption caused by osteoclasts is too high, which results in a significant loss of calcium in bones and the decrease of bone density. Therefore, if the function or activation of osteoclasts (i.e., the activity of the bone resorption) and/or the formation of osteoclasts (which can be differentiated from hematopoietic stem cells under the induction of differentiation inducers such as RANKL (receptor for activation of nuclear factor kappa B ligand) or M-CSF (macrophage colony stimulating factor)) can be inhibited, the bone resorption can be controlled to prevent or alleviate osteoporosis. Moreover, another cause for osteoporosis is that the bone formation activity of osteoblasts in bones is insufficient. Hence, if osteoblasts can be activated to stimulate bone formation, calcium in bones can be increased so as to prevent or alleviate osteoporosis.

It has been disclosed in documents that flavone components of Lespedeza plants have physiological activity, such as anti-allergy and anti-oxidation effects (see Wang et al., Evaluation of Lespedeza germplasm genetic diversity and its phylogenetic relationship with the genus Kummerowia Conserv Genet, DOI 10.1007/s10592-008-9524-2, which is entirely incorporated hereinto by reference).

As shown in the following examples, the inventors of the present invention found that the Flemingia macrophylla extract has the effects of inhibiting the activation and formation of osteoclasts, and thus may alleviate/inhibit bone resorption. Furthermore, for the first time, after purifying an active component of Flemingia macrophylla, the inventors of the present invention confirmed that the active component is Lespedeza flavanone A (LDF-A, the compound of formula (I)), and discovered that the component has activity of inhibiting osteoclasts.

Accordingly, the present invention provides a Flemingia macrophylla extract for inhibiting the formation and/or activation of osteoclasts comprising the compound of formula (I):

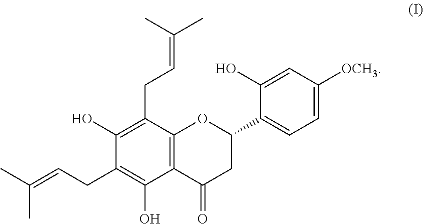

The Flemingia macrophylla extract of the present invention comprises about 0.15 wt % to about 0.35 wt % of the compound of formula (I), based on the dry weight of the extract. Preferably, the extract comprises about 0.20 wt % to about 0.30 wt % of the compound of formula (I), based on the dry weight of the extract.

Because the Flemingia macrophylla extract of the present invention has the activity of inhibiting the formation and/or activation of osteoclasts, it can achieve the anti-osteoporosis effect. In this text, the term “anti-osteoporosis” covers the prevention of osteoporosis, the improvement in osteoporosis, and the treatment of osteoporosis.

It is believed that, but not limited thereby, the mechanism of the compound of formula (I) in the Flemingia macrophylla extract used in the present invention is different from that of estrogen since antagonists of estrogen receptors cannot inhibit the activity of the compound of formula (I). The pharmacological mechanism of the Flemingia macrophylla extract of the present invention primarily resides in inhibiting the formation and/or activation of osteoclasts so as to alleviate the loss of calcium in bones of postmenopausal females caused by the decrease in estrogen due to menopause. Hence, the extract of the present invention may replace the hormone supplement therapy and reduce the side effects caused thereby, such as cancer, psychiatric diseases, etc.

The present invention also provides a method for the preparation of the Flemingia macrophylla extract, comprising extracting Flemingia macrophylla with a polar solution, and collecting the liquid phase to obtain the Flemingia macrophylla extract, wherein the polar solution comprises ethanol and water in a volumetric ratio of about 40:60 to about 100:0. Preferably, the polar solution comprises ethanol and water in a volumetric ratio of about 60:40 to about 80:20, and more preferably in a volumetric ratio of about 70:30 to about 78:22.

The extracting step in the present invention may be optionally assisted with other appropriate extracting means (e.g., ultrasonic vibration, etc.) to increase extraction efficiency. Furthermore, the method for the preparation of the Flemingia macrophylla extract may comprise conducting the extracting step two or more times (i.e., repeating the extracting step one or more times), combining the liquid phase obtained from each extracting step to provide a combined mixture, and optionally concentrating the combined mixture under a vacuum condition. Accordingly, the active components in Flemingia macrophylla can be separated from the inactive components contained in the same as much as possible to reduce the resource waste and increase economical benefit.

Depending on the application forms of the Flemingia macrophylla extract, a drying step may be optionally carried out to dry the obtained extract. For example, if the Flemingia macrophylla extract of the present invention is applied by oral administration, a drying step (such as concentrating under a vacuum condition and/or ventilation) can be used to remove organic solvents in the extract to make the obtained extract easy to apply, store, etc.

In one embodiment of the present invention, the Flemingia macrophylla extract of the present invention is obtained via the following method. First, the root and stem of Flemingia macrophylla were extracted twice with a polar solution comprising ethanol and water in a volumetric ratio of 75:25 (ethanol:water), and the liquid phase was collected. The solvent was evaporated under a reduced pressure at 50° C. to remove ethanol, and the Flemingia macrophylla extract was obtained. Based on the dry weight of Flemingia macrophylla, the yield of the extract was about 7.8 wt %.

The present invention also provides a method for inhibiting the formation and/or activation of osteoclasts in a mammal, comprising administrating an effective amount of the compound of formula (I) or a pharmaceutically acceptable salt or ester thereof to the mammal. The method of the present invention can be used for anti-osteoporosis. The compound of formula (I), Lespedeza flavanone A, in the method of the present invention can be provided from any natural or artificial sources. Herein, any suitable extraction methods can be adopted in combination with an isolation operation to obtain Lespedeza flavanone A from Lespedeza plants or Flemingia macrophylla. These isolation methods, for instance, are disclosed in Wang et al. Two flavanones from the root bark of Lespedeza davidii. Phytochemistry, 1987; 26: 1218-9, which is entirely incorporated hereinto by reference.

In one embodiment of the present invention, Lespedeza flavanone A is obtained by the following method. First, the Flemingia macrophylla extract was dissolved in the water and then partitioned with n-butanol to acquire an n-butanol extraction portion. The n-butanol extraction portion was dissolved in the water and partitioned with chloroform to acquire a chloroform extraction portion. Then, chromatography for the chloroform extraction portion was conducted with a silica gel to acquire Fractions 1 to 10. Fraction 4 was collected, and chromatography for Fraction 4 was carried out with a silica gel column to obtain Fractions 4-a to 4-h. Finally, Fraction 4-c was collected and purified with a high performance liquid chromatography (HPLC) instrument to acquire Lespedeza flavanone A.

Lespedeza flavanone A or the pharmaceutically acceptable salt or ester thereof can be administrated as a medicament or a Flemingia macrophylla extract, and the Flemingia macrophylla extract can be prepared by the method described hereinbefore. In terms of the medicament, it can be applied in any suitable way. For instance, but not limited thereby, the medicament can be applied by oral administration, subcutemeous administration, or intravenous administration, etc. The medicament can be used individually or in combination with adjuvants, and can be used in both veterinary medicine and human medicine in practice.

With respect to the manufacture of a medicament suitable for oral administration, the medicament may comprise adjuvants that do not adversely influence the activity of the compound of formula (I). For instance, the adjuvants can be a solvent, an oil solvent, a thinner, a stabilizer, an absorption-retarding reagent, a disintegrant, an emulsifier, a binder, a lubricant, a deliquescent, etc. For example, the solvent can be water or a sucrose solution; the thinner can be lactose, starch, or microcrystalline cellulose; the absorption-retarding reagent can be chitosan or glycosaminoglycan; the lubricant can be magnesium carbonate; and the oil solvent can be a plant oil or animal oil, such as olive oil, heliotrope oil, fish liver oil, etc. With a conventional method, the medicament can be made into a suitable oral administration form, such as a tablet, a capsule, a granule, a powder, a fluid extract, a solution, a syrup, a suspension, an emulsion, a tincture, etc.

As for a medicament suitable for a subcutemeous or intravenous administration form, the medicament may comprise one or more adjuvants, such as a solubilizer, an emulsifier, or others, to produce an intravenous injection, an emulsion intravenous injection, an injection, a powder injection, a suspension injection, a powder-suspension injection, etc. And the solvent for the injections can be selected from the following group: water, a saline solution, alcohols (e.g., ethanol, propanol, glycerin, etc), sugar solutions (e.g., a glucose or mannitol solution), or combinations thereof.

Optionally, in addition to the above adjuvants, other additives, such as a flavoring agent, a toner, a coloring agent, etc., may be also added into the medicament to enhance the sense of comfort for the mouth and visual feelings during the administration. A suitable amount of a preservative, a conservative, an antiseptic, an anti-fungus reagent, etc., can also be added to improve the storability of the medicament.

Moreover, the medicament may be optionally incorporated with one or more other active components such as alendronate, parathorine, estrogen, calcium compounds, or Vitamin D, and the likes to enhance the effects of the medicament or increase the flexibility and plasticity of the formulation, as long as the other active components have no adverse effects on the compound of formula (I).

Depending on the needs of the subjects to be treated, the medicament can be applied with various administration frequencies, such as once a day, several times a day, or once for several days, etc. For example, if the medicament is used for anti-osteoporosis, the amount of the medicament administrated to a mammal, calculated as the compound of formula (I), is about 0.06 mg/kg-body weight to about 1.93 mg/kg-body weight per day, wherein the unit “mg/kg-body weight” refers to the amount of the medicament needed for the administration per kg-body weight. Preferably, the amount of the medicament administrated to a mammal, calculated as the compound of formula (I), is about 0.08 mg/kg-body weight to about 1.65 mg/kg-body weight per day. However, in an acute situation (e.g., serious osteoporosis), the dosage can be increased to several times or several tens of times, depending on the practical needs.

The present invention also provides a use of the compound of formula (I) or the pharmaceutically acceptable salt or ester thereof or the above Flemingia macrophylla extract in the manufacture of a medicament, wherein the medicament may be manufactured in any suitable form for inhibiting the formation and/or activation of osteoclasts. In the use of the manufacture of a medicament, the details regarding the components and the administration are described in the above description.

The present invention will be further illustrated in details with specific examples as follows. After referring to the examples described in the following paragraphs, people skilled in this field can easily appreciate the basic spirit and other invention purposes of the present invention, and technical methods adopted in the present invention and better embodiments. However, the following examples are provided only for illustrating the present invention, and the scope of the present invention is not limited thereby.

### Example 1

### Preparation of the Flemingia macrophylla Extract

Flemingia macrophylla was purchased from a market in Taichung, Taiwan. The specimen of this plant has been deposited in the College of Pharmacy, China Medical University, Taiwan, and has been identified by the college. The root and stem of Flemingia macrophylla were extracted with a polar solution comprising ethanol and water in a volumetric ratio of 75:25 for twice, and a portion soluble in a liquid phase was collected, and under a reduced pressure at 50° C., the solvent was evaporated to remove ethanol, and a Flemingia macrophylla extract was prepared. The yield of the prepared Flemingia macrophylla extract, based on the dry weight of Flemingia macrophylla, was about 7.8 wt %.

### Example 2

### Animal Tests

Female Wistar rats (BioLASCO, Taipei, Taiwan) were used in the following experiments. In an experiment group, the rats were anesthetized with 40 mg/kg-body weight of pentobarbital sodium, and ovaries in both sides of the dorsum of the rats were removed to induce osteoporosis, and a decrease in estrogen of the postmenopausal female rats was observed. The rats of which ovaries were removed (i.e., ovariectomized rat) are represented as “OVX rats” hereinafter. In the control group (i.e., a sham group), the skin and muscle on the ovary positions at both sides of the dorsum of the rats were incised and then sutured, while the ovaries were not removed.

After one week from the surgery, the control group was administrated water orally every day. In the experiment group, the OVX rats were separated into five groups, and were orally administrated with the Flemingia macrophylla extract (0, 50, 250 or 500 mg/kg-body weight, calculated as the dry weight of the extract) or 2.5 mg/kg-body weight of alendronate (an anti-osteoporosis drug as a positive control, Sigma-Aldrich, St. Louis, Mo., USA) per day for 13 weeks. During the administration, the body weight of each rat was measured once a week until the last day of the administration. Distilled water was added into the extract to form a suspension, and the rats were administrated orally with 1 ml/100 g-body weight of the suspension.

### Example 3

### The Observation of the Change of the Body Weight and Vagina Weight of the Rats

After the end of the administration in Example 2, the rats were anesthetized with a high dosage of pentobarbital sodium (65 mg/kg-body weight) and were sacrificed. Blood samples of the rats were then collected, and tibiae and vertebrae of the rats were dissected, and vaginas were taken out from the rats and the weight of the vaginas was measured. The results are shown in Table 1.

TABLE 1

| | dosage | | average body weight | |
|---|---|---|---|---|
| | (mg/kg-body | average body weight | after 13 weeks from | average vagina |
| Group | weight) | before surgery (g) | the surgery (g) | weight (g) |
| Control | water | 267.1 ± 13.6 | 289.0 ± 19.0 | 0.50 ± 0.04 |
| Experiment | extract, | 260.0 ± 22.7 | 372.8 ± 30.7 | 0.17 ± 0.02^{###} |
| group 1 | 0 | | | |
| Experiment | extract | 263.7 ± 18.4 | 378.8 ± 34.2 | 0.18 ± 0.02 |
| group 2 | 50 | | | |
| Experiment | extract | 263.8 ± 28.0 | 369.1 ± 27.6 | 0.17 ± 0.03 |
| group 3 | 250 | | | |
| Experiment | extract | 260.3 ± 18.0 | 374.8 ± 25.7 | 0.17 ± 0.02 |
| group 4 | 500 | | | |
| Positive | alendronate | 266.6 ± 20.1 | 377.8 ± 36.5 | 0.18 ± 0.01 |
| control | 2.5 | | | |

Compared to the control group, ^{###}P < 0.001.

As can be seen in Table 1, after the surgery, the body weight of the ovariectomized rats (i.e., OVX rats) increased apparently, and the body weight of the ovariectomized rats administrated with the Flemingia macrophylla extract (OVX rats+the extract) did not increase apparently. Furthermore, after the ovaries were removed, the vagina weight of the rats decreased, showing that the Flemingia macrophylla extract could not efficiently improve the decrease of the vagina weight of the rats. It is known that estrogen can inhibit the increase of the body weight and the decrease of the vagina weight of the rats. This experiment shows that after the rats were ovariectomized, the increase of the body weight and the decrease of the vagina weight (vagina atrophy) may occur, and the Flemingia macrophylla extract cannot inhibit the increase of the body weight or the decrease of the vagina weight of the rats efficiently, and thus, the Flemingia macrophylla extract does not have the same effect as estrogen.

### Example 4

### Measurement of the Density and Content of Minerals in Bones of the Rats

A dual-energy X-ray absorptiometer (XR-26, Norland, Fort Atkinson, Wis., USA) was used to measure the density and content of minerals in the tibiae of the rats in Example 3, and the results are shown in Table 2.

TABLE 2

| | dosage | | |
|---|---|---|---|
| | (mg/kg-body | mineral content | mineral density |
| Group | weight) | (g) | (g/cm³) |
| Control | — | 0.44 ± 0.01 | 0.14 ± 0.01 |
| OVX + water | — | 0.31 ± 0.02^{###} | 0.12 ± 0.01^{###} |
| OVX + extract | 50 | 0.33 ± 0.01* | 0.12 ± 0.01 |
| OVX + extract | 250 | 0.40 ± 0.01*** | 0.13 ± 0.00* |
| OVX + extract | 500 | 0.39 ± 0.01*** | 0.14 ± 0.01*** |
| OVX + alendronate | 2.5 | 0.38 ± 0.01*** | 0.14 ± 0.01*** |

All data are mean ± standard deviation (the number of samples = 8).
Compared to the control group, ^{###}P < 0.001.
Compared to the OVX + water group, *P < 0.05, ***P < 0.001.

As can be seen in Table 2, compared to the control group, under the condition in which the OVX rats were not administrated with the Flemingia macrophylla extract, the density and content of minerals in the tibiae of the OVX rats decreased by 14.3% and 29.5%, respectively. After the OVX rats were administrated with the Flemingia macrophylla extract, both the density and content of minerals in the tibiae of the OVX rats increased apparently, and thus the Flemingia macrophylla extract can inhibit the loss of minerals in bones.

### Example 5

### Measurement of the Weight of Bone Ash and the Calcium Content of the Rats

The fifth sections of the vertebrae of the rats in Example 3 were dissected in an ethanol bath and dried at 100° C. overnight, and the dry weight of the vertebrae was measured. Then, the vertebrae were cremated at 1000° C. for 12 hours. After the cremation, bone ash was collected, and the weight ratio of the bone ash (represented as the percentage of the dry weight of the vertebrae) was measured.

Thereafter, the bone ash was dissolved in 6N hydrochloric acid, and the calcium content in the bone ash (denoted as “mg/cm³ bone volume”) was measured according to Archimedes' method (see Kalu, The ovariectomized rat model of postmenopausal bone loss, Bone Miner, 1991: 15, 175-91, which is entirely incorporated hereinto by reference). The results are shown in Table 3.

TABLE 3

| | dosage | the weight | |
|---|---|---|---|
| | (mg/kg-body | ratio of | calcium content |
| Group | weight) | bone ash (%) | (mg/cm³) |
| Control | — | 64.5 ± 8.1 | 209.7 ± 26.8 |
| OVX + water | — | 54.4 ± 3.8^{##} | 171.7 ± 8.9^{##} |
| OVX + extract | 50 | 58.9 ± 7.1 | 186.9 ± 25.8 |
| OVX + extract | 250 | 62.6 ± 3.1* | 198.3 ± 14.2 |
| OVX + extract | 500 | 63.9 ± 5.2** | 205.6 ± 26.2* |
| OVX + alendronate | 2.5 | 62.1 ± 1.5* | 206.1 ± 24.2* |

All data are mean ± standard deviation (the number of samples = 8).
Compared to the control group, ^{##}P < 0.01.
Compared to the OVX + water group, *P < 0.05, **P < 0.01.

As can be seen in Table 3, compared to the control group, under the condition in which OVX rats were not administrated with the Flemingia macrophylla extract, the weight ratio of the bone ash and the calcium content of the OVX rats decreased by 15.7% and 18.1%, respectively, and the weight ratio of the bone ash and the calcium content of the OVX rats administrated with the extract increased apparently. Accordingly, this experiment shows that the Flemingia macrophylla extract can inhibit the loss of bone components and calcium.

### Example 6

### Tissue Analysis of the Tibiae of the Rats

The left tibiae of the rats in Example 3 were taken and fixed with 4 vol % metaformaldehyde (in a phosphate buffer solution, PBS, pH 7.4) for 48 hours, and calcium was removed in a solution (pH 7.4) of 10 wt % EDTA (ethylenediamine tetraacetic acid) at 4° C. for 4 weeks. After calcium was removed, the bones were cut to generate a cross-section, and tissue staining was carried out with hematoxylin and eosin. After staining, the change of microstructure of the cross-section was observed with a microscope. The microscope images were analyzed with a color image analysis system (Image-Pro Plus version 5.1, Media Cybernetics, Maryland, USA), and the volume of trabecular bones and the thickness of dermal bones of metaphysis of the rats were measured. The volume of the trabecular bones is represented as “the percentage of the whole tissue area at the sampling position.”

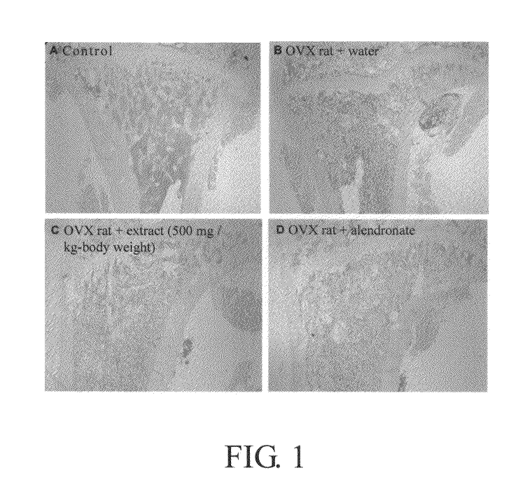

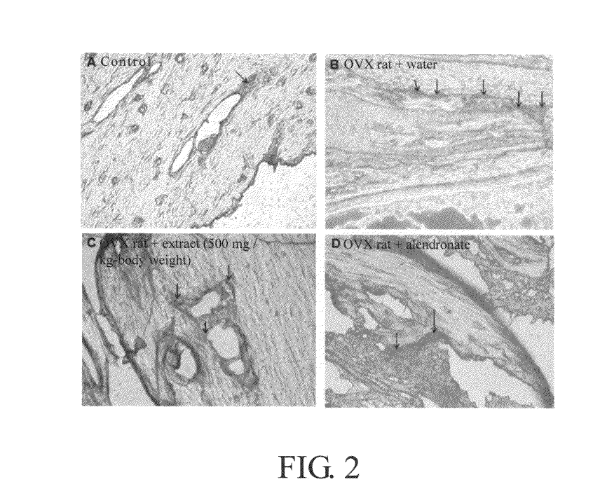

In another aspect, the above cross-section was stained with a TRAP (tartrate-resistant acid phosphatase) kit (Sigma-Aldrich, St. Louis, Mo., USA), and the number of osteoclasts was measured (see Cole et al., Tartrate-resistant acid phosphatase in bone and cartilage following decalcification and cold-embedding in plastic. J Histochem Cyrochem, 1987: 35, 203-6, which is entirely incorporated hereinto by reference). The results are shown in FIGS. 1 and 2 and Table 4.

TABLE 4

| | dosage | trabecular bone | | osteoclast |
|---|---|---|---|---|
| | (mg/kg-body | volume/tissue | thickness of dermal | number/bone |
| Group | weight) | volume (%) | bones (μm) | surface (mm) |
| Control | — | 38.9 ± 5.0 | 214.5 ± 30.4 | 1.3 ± 0.4 |
| OVX + water | — | 30.6 ± 4.0^{##} | 169.8 ± 13.9^{###} | 4.8 ± 0.4^{###} |
| OVX + extract | 50 | 34.9 ± 3.3 | 182.3 ± 14.5 | 4.1 ± 1.0 |
| OVX + extract | 250 | 36.2 ± 3.7* | 200.5 ± 5.7** | 2.9 ± 0.8*** |
| OVX + extract | 500 | 36.3 ± 1.4** | 215.9 ± 13.3*** | 2.7 ± 0.3*** |
| OVX + alendronate | 2.5 | 36.1 ± 1.1** | 215.0 ± 10.2** | 2.7 ± 0.4*** |

All data are mean ± standard deviation (the number of samples = 8).
Compared to the control group, ^{##}P < 0.01, ^{###}P < 0.001.
Compared to the OVX + water group, *P < 0.05, **P < 0.01.

As shown in FIGS. 1 and 2 and Table 4, compared to the OVX rats that were not administrated with the extract, it is obvious that the volume of the trabecular bones and the thickness of the dermal bones of the OVX rats administrated with the extract are greater, which indicates that the extract used in the present invention can efficiently inhibit the decrease of the thickness of the dermal bones and the volume of the trabecular bones due to the insufficiency of estrogen. Furthermore, the results of this experiment also show that the Flemingia macrophylla extract can reduce the number of osteoclasts at primary spongiosa area to inhibit bone resorption.

### Example 7

### Measurement of Biochemical Values of the Blood and Urine of the Rats

The activity of ALP (alkaline phosphatase) and the content of calcium and creatinine in the blood and urine of the rats were measured with a clinical trial kit (Roche Diagnostics, Mannheim, Germany) and a spectrophotometric analyzer (Cobas Mira Plus, Roche, Rotkreuz, Switzerland), and the calcium content in the urine and blood of the rats was also measured with a method using o-cresolphthalein complexone (Randox, UK). ALP is a bio-indicator or molecular marker of an early activation stage of preosteoblasts, and based on ALP activity, the activation situation of preosteoblasts can be observed. The calcium content in the urine is represented as “mg/mmol creatinine in the urine.”

Moreover, the content of deoxypyridinoline (DPD) in the urine was measured with ELISA (enzyme linked immunosorbent assay) (an ELISA kit is purchased from Metra, San Diego, Calif., USA), and is represented as “mg/mmol creatinine in the urine.” The results are shown in Table 5.

TABLE 5

| | | | calcium content | DPD content in |
|---|---|---|---|---|
| | dosage | | in urine | urine |
| | (mg/kg-body | ALP | (mg/mmol | (mg/mmol |
| Group | weight) | (U/L) | creatinine) | creatinine) |
| Control | — | 120.6 ± 24.6 | 39.7 ± 21.0 | 292.4 ± 58.7 |
| OVX + water | — | 167.1 ± 25.0^{##} | 89.3 ± 40.3^{##} | 520.7 ± 97.0^{###} |
| OVX + extract | 50 | 160.9 ± 38.5 | 52.5 ± 12.4 | 444.7 ± 104.2 |
| OVX + extract | 250 | 156.3 ± 12.5 | 39.3 ± 20.3* | 384.5 ± 106.2* |
| OVX + extract | 500 | 150.6 ± 20.0 | 37.3 ± 15.3** | 380.4 ± 69.5* |
| OVX + alendronate | 2.5 | 163.5 ± 24.0 | 45.2 ± 14.8** | 281.2 ± 52.3*** |

All data are mean ± standard deviation (the number of samples = 8).
Compared to the control group, ^{###}P < 0.001.
Compared to the OVX + water group, *P < 0.05, **P < 0.01, ***P < 0.001.

In this experiment, the activity of ALP in the blood serum of the rats was used as a marker of bone formation (see Swaminathan, Biochemical markers of bone turnover, Clin Chim Acta, 2001: 313, 95-105, which is entirely incorporated hereinto by reference), and the content of DPD in the urine of the rats was used as a marker of the bone resorption (see Seibel et al., Urinary hydroxypyridium crosslinks of collagen as makers of bone resorption and estrogen efficacy in postmenopausal osteoporosis. J Bone Miner Res., 1993: 8, 881-9, which is entirely incorporated hereinto by reference).

As can be seen in Table 5, the OVX rats that were not administrated with the Flemingia macrophylla extract have higher ALP activity and content of calcium and DPD in the urine, which indicates that after the ovaries were removed from the rats, the insufficiency of estrogen promoted bone resorption and caused the loss of calcium to initiate the bone formation.

In addition, the OVX rats that were administrated with the Flemingia macrophylla extract had lower activity of bone formation and bone resorption, and as the OVX rats were administrated with 250 mg/kg-body weight or 500 mg/kg-body weight of the extract, the concentration of calcium in the urine of the OVX rats was much lower than that of the rats administrated with alendronate, which indicates that the extract used in the present invention can efficiently improve the loss of calcium in bones.

Furthermore, because the Flemingia macrophylla extract can reduce the DPD content in the urine, but does not affect the activity of ALP, it can be inferred that the Flemingia macrophylla extract achieves the anti-osteoporosis effect via inhibition of bone resorption, not via promotion of bone formation.

### Example 8

### Purification of Active Components of the Flemingia macrophylla Extract

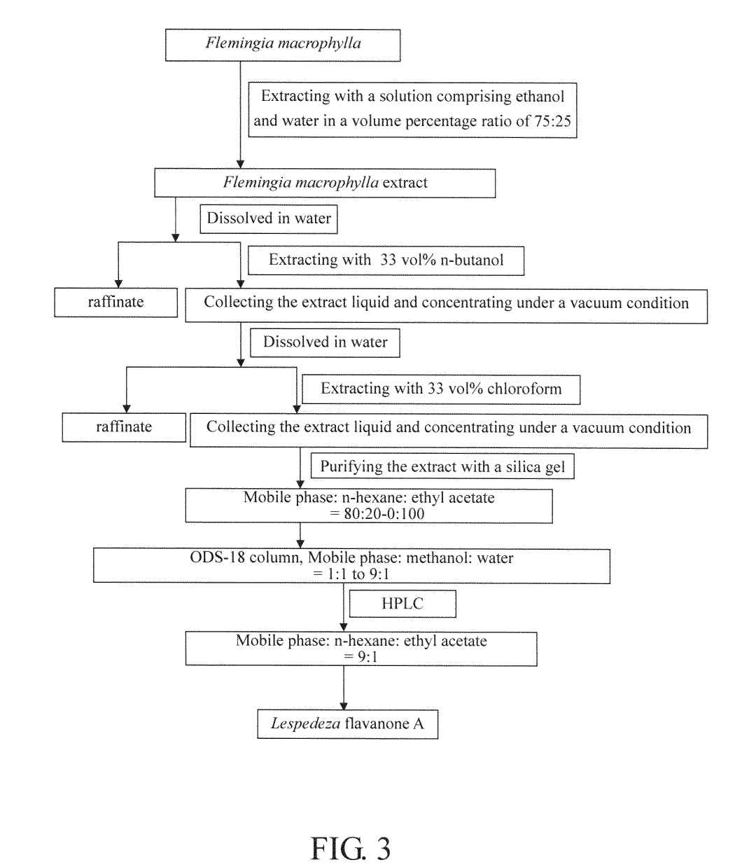

The main active component of the Flemingia macrophylla extract was purified according to the steps shown in FIG. 3. First, the prepared Flemingia macrophylla extract in Example 1 was dissolved in water to form a suspension, and then the suspension was partitioned with 33 vol % n-butanol (n-butanol:water=1:2, volume ratio) to obtain an n-butanol extraction portion, and the extraction portion was concentrated, the yield of which was 2.8 wt %.

Then, 75 g n-butanol extraction portion was dissolved in water and partitioned with 33 vol % chloroform (chloroform:water=1:2, volume ratio) to acquire a chloroform extraction portion, the weight of which after drying was 21.6 g. Chromatography for the chloroform extraction portion was then conducted with a silica gel (Si 60 F245, Merck, Germany) (the mobile phase is n-hexane/ethyl acetate, 80/20 to 0/100, volume ratio) to acquire Fractions 1 to 10.

Fraction 4 (6.1 g) was collected and chromatography for Fraction 4 was conducted with a silica gel column (ODS-18 Column, LiChroprep RP-18, Merck) (the mobile phase is methanol/water, 1/1 to 9/1, volume ratio) to acquire Fractions 4-a to 4-h. Fraction 4-c (370 mg) was then purified with a high performance liquid chromatography (HPLC) instrument to acquire a purified compound (75 mg). The conditions of the liquid phase chromatography are as follows. Pump: Shimadzu LC-8A (Kyoto, Japan); mobile phase: n-hexane/ethyl acetate (9/1, volume ratio); column: PEGAAIL Silica 60-5 (inner diameter: 10 mm, length: 250 mm, Senshu Scientific Co. Ltd., Tokyo, Japan).

The purified compound was identified with a mass spectrometer (HP 5995C, USA) and a nuclear magnetic resonance (NMR) instrument (¹H, ¹³C, DEPT, COXY, HMQC, HMBC, Bruker 400 MHz, Germany), and was confirmed as Lespedeza flavanone A (i.e., LDF-A, the compound of formula (I)), the IUPAC name of which is 5,7,2-trihydroxy-6,8-di-r,r-dimethylallyl-4-methoxyflavanone. Table 6 shows the NMR data of Lespedeza flavanone A.

TABLE 6
¹³C and ¹H NMR data of Lespedeza flavanone A
(400 MHz, acetone-d₆)

| C | | H |
|---|---|---|
| C-2 | 74.35 | 5.68 (dd, 12.8. 2.9) |
| C-3 | 41.86 | 3.09 (dd, 16.8. 12.9) 2.79 (dd, 17.1. 2.9) |
| C-4 | 197.42 | — |
| C-5 | 160.85 | — |
| C-6 | 107.11 | — |
| C-7 | 159.33 | — |
| C-8 | 107.80 | — |
| C-9 | 161.42 | — |
| C-10 | 102.44 | — |
| C-1′ | 118.24 | — |
| C-2′ | 155.31 | — |
| C-3′ | 105.00 | 6.52 (d, 2.1) |
| C-4′ | 158.44 | — |
| C-5′ | 101.48 | 6.52 (dd, 9.3. 2.4) |
| C-6′ | 127.73 | 7.43 (d, 6.8) |
| C-1″ | 20.06 | 3.31 (d, 7.4) |
| C-2″ | 122.65 | 5.17 (m) |
| C-3″ | 131.28 | — |
| C-4″ | 17.07 | 1.61 (s) |
| C-5″ | 25.01 | 1.62 (s) |
| C-1′″ | 20.94 | 3.33 (d, 7.1) |
| C-2′″ | 122.51 | 5.19 (m) |
| C-3′″ | 131.07 | — |
| C-4′″ | 17.07 | 1.65 (s) |
| C-5′″ | 25.01 | 1.75 (s) |
| C-4′-OCH3 | 54.63 | 3.76 |

The content of Lespedeza flavanone A was determined with an HPLC instrument. The conditions of the liquid phase chromatography are as follows. Pump: Shimadzu LC-6A; UV spectrophotometer detector: SPD-6AV; column: Cosmosil 5C18-AR-II Column (inner diameter: 4.6 mm, length: 250 mm, Nacalai Tesque Inc., Tokyo, Japan); mobile phase: two elution solutions, water and 0.2 wt % acetic acid or acetonitrile (linear gradient, 0 minute: 30 vol % acetic acid; 17^{th} minutes: 20 vol % acetic acid; 40^{th} minutes: 20 vol % acetic acid), flow rate: 0.3 ml/minute; UV detection with a wavelength of 270 nm. The results show that the content of Lespedeza flavanone A in the Flemingia macrophylla extract was 0.26 wt %.

### Example 9

### Differentiation Tests of Osteoclasts

Bone marrow was taken out from the femurs and tibiae of Wistar rats, and bone marrow cavities were washed with a α-minimum essential medium (Hyclone, Logan, Utah, USA) comprising a 20 mmol HEPES buffer solution, 10 vol % heat-inactivated fetal bovine serum, 2 mmol glutamine, 100 U/ml penicillin, and 100 g/ml streptomycin. After 24 hours, inadhesive hematopoietic cells that were washed were collected, and were used as osteoclast precursors.

The aforesaid precursor cells were incubated in 24-wells culture plates with a density of 1×10⁶ cells/well, and the culture plates comprised soluble human recombinant RANKL (50 ng/ml, PeproTech EC, London, UK) and M-CSF (20 ng/ml, PeproTech EC). Under the induction of M-CSF and RANKL, the precursor cells may differentiate into osteoclasts.

Then, various concentrations of the Flemingia macrophylla extract or Lespedeza flavanone A were added into the culture plates, and culture medium was replaced once every 3 days. On the ninth day, osteoclasts were stained with a TRAP staining kit, and the formation of osteoclasts was observed. The cells were then fixed with 10 vol % formaldehyde (in a phosphate buffer solution) for three minutes, and were stained with Naphthol AS-Mx phosphate and a tartaric acid solution at 37° C. for 1 hour. TRAP-positive multinucleated osteoclasts with more than three nuclei were regarded as osteoclasts (see Asagiri et al., The molecular understanding of osteoclast differentiation, Bone, 2007, 40: 251-64, which is entirely incorporated hereinto by reference).

To clarify the mechanism of Lespedeza flavanone A, an experiment was carried out with 1 nM ICI 182,780 (an antagonist of estrogen receptors) (see Howell et al., ICI 182,780 (Faslodex): development of a novel, “pure” antiestrogen, Cancer, 2000, 89: 817-825, which is entirely incorporated hereinto by reference), and influence of ICI 182,780 on the formation of osteoclasts was observed, and 10 nM 17β-estradiol was used for comparison. The results are shown in Table 7.

TABLE 7

the percentage of
osteoclast formation (%)

| | | | | ICI 182,780 |
|---|---|---|---|---|
| Group | concentration | | | (1 nM) |
| Control | 0 | μg/ml | 100 | |
| extract | 100 | μg/ml | 58.3 ± 7.1* | |
| extract | 300 | μg/ml | 27.6 ± 5.6*** | |
| extract | 500 | μg/ml | 14.5 ± 7.9*** | |
| Control | 0 | μM | 100 | |
| LDF-A | 0.1 | μM | 70.7 ± 9.5** | 65.0 ± 11.5 |
| LDF-A | 1.0 | μM | 60.0 ± 5.2*** | 49.0 ± 6.2 |
| LDF-A | 10.0 | μM | 27.0 ± 2.6*** | 26.0 ± 8.9 |
| 17β-estradiol | 10.0 | nM | 57.7 ± 8.1*** | 83.0 ± 13.2* |

All data are mean ± standard deviation (the number of samples = 3).
Compared to the control group, *P < 0.05, **P < 0.01, ***P < 0.001.
Compared to the 17β-estradiol group, ^{#}P < 0.05.

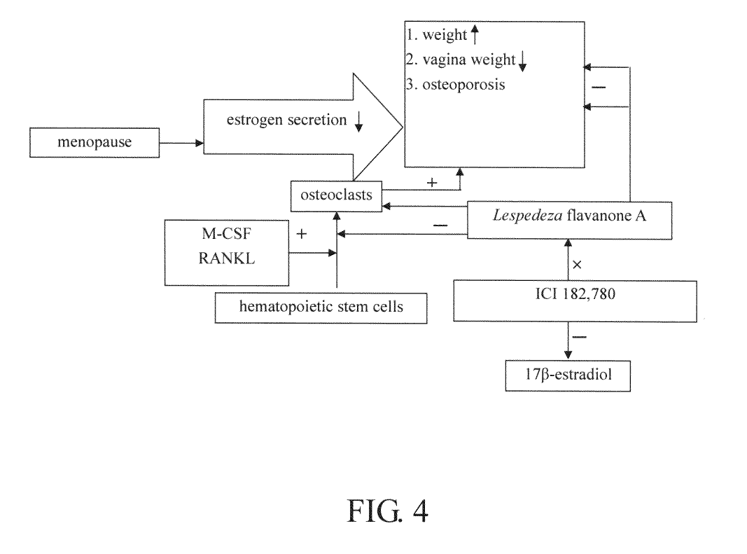

As can be seen in Table 7, the higher the dosage of the Flemingia macrophylla extract or Lespedeza flavanone A is, the more apparent their effects of inhibiting the formation of osteoclasts. Furthermore, as osteoclasts were treated with 1 nM ICI 182,780 and estradiol at the same time, ICI 182,780 clearly inhibited the inhibition effect of estradiol on the osteoclast formation. However, ICI 182,780 did not inhibit the inhibition effect of Lespedeza flavanone A on the osteoclast formation, and thus, suggests that the pathway of the inhibition effect on the osteoclast formation of Lespedeza flavanone A is not via estrogen receptors; that is, the mechanism of Lespedeza flavanone A is different from that of estrogen (see FIG. 4).

The results of the osteoclast differentiation experiment suggest that Lespedeza flavanone A is the main active component of the Flemingia macrophylla extract for anti-osteoporosis.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can make various changes and modifications of the invention to adapt it to various usages and conditions without departing from the spirit and scope thereof. Therefore, the scope of protection of the subject invention is substantially covered in the following claims as appended.

What is claimed is:

1. A method for inhibiting the formation and for activation of osteoclasts in a mammal comprising administrating an effective amount of a compound of formula (I) or a pharmaceutically acceptable salt or ester thereof to the mammal:

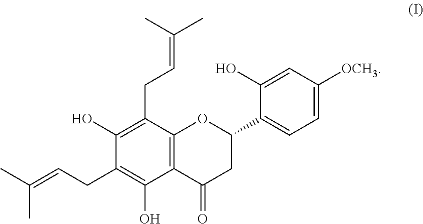

2. The method as claimed in claim 1, wherein the compound of formula (I) is from Flemingia macrophylla.

3. The method as claimed in claim 1, which is for anti-osteoporosis.

4. The method as claimed in claim 1, wherein the amount of the compound of formula (I) or the pharmaceutically acceptable salt or ester thereof administrated to the mammal, calculated as the compound of formula (I), is about 0.06 mg/kg-body weight to about 1.93 mg/kg-body weight per day.

5. The method as claimed in claim 4, wherein the amount of the compound of formula (I) or the pharmaceutically acceptable salt or ester thereof administrated to the mammal, calculated as the compound of formula (I), is about 0.08 mg/kg-body weight to about 1.65 mg/kg-body weight per day.

6. The method as claimed in claim 1, wherein the compound of formula (I) or the pharmaceutically acceptable salt or ester thereof is administrated as a medicament.

7. The method as claimed in claim 1, wherein the compound of formula (I) is administrated as a Flemingia macrophylla extract.

8. The method as claimed in claim 7, wherein the Flemingia macrophylla extract is prepared by a method comprising extracting Flemingia macrophylla with a polar solution, and collecting a liquid phase obtained from the extracting step to obtain the Flemingia macrophylla extract, wherein the polar solution comprises ethanol and water in a volumetric ratio of about 40:60 to about 100:0.

9. The method as claimed in claim 8, wherein the polar solution comprises ethanol and water in a volumetric ratio of about 60:40 to about 80:20.

10. The method as claimed in claim 9, wherein the polar solution comprises ethanol and water in a volumetric ratio of about 70:30 to about 78:22.

11. The method as claimed in claim 8, wherein the method for the preparation of the Flemingia macrophylla extract further comprises repeating the extracting step one or more times, combining liquid phases obtained from the extracting steps to provide a combined mixture, and optionally concentrating the combined mixture under a vacuum condition.

12. The method as claimed in claim 8, wherein the Flemingia macrophylla extract comprises about 0.15 wt % to about 0.35 wt % of the compound of formula (I), based on the dry weight of the extract.

13. The method as claimed in claim 12, wherein the Flemingia macrophylla extract comprises about 0.20 wt % to about 0.30 wt % of the compound of formula (I), based on the dry weight of the extract.
